(19) United States
(12) Patent Application Publication
(10) Pub. No.: US 2010/0055723 A1
(43) Pub. Date: Mar. 4, 2010

(54) Galectin-3-Binding Protein as a Biomarker of Cardiovascular Disease

(75) Inventors: David M. Smalley, Hampden, ME (US); Christian A. Gleissner, Heidelberg (DE); Klaus F. Ley, La Jolla, CA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 12/523,928

(22) PCT Filed: Jan. 23, 2008
(86) PCT No.: PCT/US08/51801

§ 371 Date: Jul. 21, 2009

Related U.S. Application Data
(60) Provisional application No. 60/881,871, filed on Jan. 23, 2007.
(60) Provisional application No. 60/994,725, filed on Sep. 21, 2007.

(51) Int. Cl. G01N 33/53

(52) U.S. Cl. 435/7.92; 435/7.1; 436/86

(57) ABSTRACT

The present invention provides compositions and methods for using biomarkers to diagnose and monitor cardiovascular associated diseases and disorders. More specifically, the present invention provides Galectin-3-binding protein as a biomarker for disease.

### CROSS REFERENCE TO RELATED APPLICATIONS

This application is entitled to priority pursuant to 35 U.S.C. §119(e) to U.S. provisional patent application Nos. 60/881,871, filed on Jan. 23, 2007, and 60/994,725, filed on Sep. 21, 2007. The entire disclosures of each of the afore-mentioned patent applications are incorporated herein by reference.

### STATEMENT REGARDING FEDERALLY SPONSORED RESEARCH OR DEVELOPMENT

This invention was supported in part from Grant No. HL58108 awarded by the National Institutes of Health. The United States Government has certain rights in the invention.

### BACKGROUND

Plasma is one of the most complex and useful human proteomes. Detection of proteins within this type of sample is currently an important tool for evaluating the predisposition, presence, and progression of numerous clinical conditions. However, the current methodology of detecting and measuring individual proteins only begins to scratch the surface of its full potential. Today, tests for only about 120 different protein analytes have been approved by the FDA and approval of tests to detect new protein analytes has averaged only about one test per year over the last decade. This represents only an extremely small fraction of the 40,000 different proteins present in the normal sample and another 500,000 proteins which may be present under a variety of clinical conditions. This has led investigators to attempt to develop methods using high-throughput detection methods to identify many more proteins in the plasma. Current methodology, based on 2D gels, liquid chromatography, and/or mass spectrometry, has lead to the detection of about 500 different plasma proteins. However, this appears to be the limit of detection for the analysis of unfractionated plasma due to the limited dynamic range of the methods used. Abundant proteins, such as albumin (35-45 mg/ml), fibrinogen (2-6 mg/ml), IgG (12-18 mg/ml), and transferrin (2-3 mg/ml), interfere with the detection of proteins that may be present at up to 10 orders of magnitude lower concentrations. Removal of the ten most abundant proteins only increases the sensitivity by one order of magnitude. This has led some to attempt to fraction the plasma samples to reduce its complexity prior to analysis.

There is a long felt need in the art for compositions and methods useful for identifying biomarkers of disease. The present invention satisfies this need.

### BRIEF SUMMARY OF INVENTION

The present invention discloses, inter alia, that Galectin 3-binding protein (G3BP) is useful as a biomarker and that it is a macrophage-derived protein.

G3BP protein is also known as Mac2BP. It is a secreted glycoprotein that binds Mac-2, a soluble lectin. G3BP belongs to the family of scavenger receptor cysteine-rich domain proteins. It may have functions in cell adhesion and multicellular aggregation. G3BP has been implicated in macrophage migration, immune response modulation, and metastasis. Elevated levels are detected in serum of tumor patients (breast, lung, colon, ovary, pancreatic carcinoma) and involved in progression and metastasis. Elevated levels have also been found in viral infections (HCV and HIV). G3BP can be detected in blood, urine, milk, semen, urine. It binds to galectin-1, -3, and -7 and to collagen type IV and V. If used as an adhesive substrate, G3BP reduces apoptosis of Jurkat T cells. G3BP promotes NK-cell generation from PBMC and downregulates Th2 cytokines in asthma. G3BP is expressed in synovia of rheumatoid arthritis patients.

For the invention disclosed herein, we first used a method to reduce the complexity of proteins present in plasma by first isolating plasma-derived microparticles, leading to the identification of 26 proteins that are uniquely expressed or significantly over-expressed in plasma-derived microparticles and not in platelet-derived microparticles as candidate biomarkers.

To identify one or more candidate biomarkers for cardiovascular disease, we used Affymetrix gene chip analysis, an independent method that is completely unrelated to mass spectrometry. Human blood-derived monocytes were cultured for several days with M-CSF or platelet factor 4 to generate macrophages. These macrophages were differentiated to foam cells using oxidized low density lipoprotein (LDL), minimally modified LDL or native LDL. We reasoned that a molecule that could be detected in human plasma (mass spectrometry) and was expressed in a cell type relevant to cardiovascular disease (macrophage-derived foam cells) would be a good candidate for a robust biomarker. Beyond the biomarkers identified so far, the methods disclosed herein demonstrate that they can also be used to identify additional biomarkers from human blood. Therefore, the present invention further encompasses the use of biomarkers described herein, as well as those identified using the methods of the invention, are useful for identifying disease states in patients, monitoring progression of disease and responsiveness to treatment.

Upon activation, many different cell types release microparticles. It is likely that composition and number of microparticles in the plasma may be important markers for disease predisposition, diagnosis, and progression. The protein identified here as differentially expressed in plasma-derived but not platelet-derived microparticles, G3BP, is induced in human macrophages by oxidized LDL. Therefore, it likely represents a plasma biomarker of vascular macrophage foam cells, which are known to be most abundant in vulnerable atherosclerotic plaques that are prone to rupture and thus precipitate heart attacks, strokes, or other events.

Therefore, the present invention provides compositions and methods useful for diagnosing a cardiovascular associated disease or disorder in a test subject, comprising obtaining a biological sample from the test subject, and comparing the level of galectin 3-binding protein in the sample with the level of galectin 3-binding protein in an otherwise identical biological sample from a control subject without the cardiovascular associated disease or disorder. A different level of galectin 3-binding protein in the sample obtained from the test subject, compared with the level of galectin 3-binding protein in the biological sample from the control subject, is an indication that the test subject has a cardiovascular associated disease or disorder.

In one embodiment, the cardiovascular associated disease or disorder is selected from the group consisting of coronary artery disease, circulatory disease exacerbated by ischemia, atherosclerosis, peripheral vascular disease, restenosis following angioplasty, surgical revascularization, inflammatory aortic aneurysm, vasculitis, stroke, spinal cord injury, congestive heart failure, cardiomyopathy, hemorrhagic shock, ischemia/reperfusion injury, vasospasm following subarachnoid hemorrhage, vasospasm following cerebrovascular accident, pleuritis, pericarditis, and the cardiovascular complications of diabetes. In one aspect, the cardiovascular associated disease or disorder is coronary artery disease. In one aspect, the test subject is a human. In one aspect, the test subject is at risk for the cardiovascular associated disease or disorder. In one aspect, the test subject is asymptomatic for said cardiovascular associated disease or disorder. In one aspect, a subject who is asymptomatic includes a subject who does not present with angina.

One of ordinary skill in the art will appreciate that various biological samples can be used. In one aspect, the sample is selected from the group consisting of tissue, cells, blood, plasma, serum, tears, saliva, feces, semen, milk, sweat, and urine.

In one aspect, the sample is plasma. In one aspect, the plasma is processed to obtain plasma-derived microparticles.

One of ordinary skill in the art will appreciate that additional biomarkers can be measured in addition to G3BP.

In one embodiment, the change in galectin 3-binding protein levels found in a test subject is an increase in galectin 3-binding protein levels.

In another embodiment, the change in galectin 3-binding protein levels in a test subject is a decrease in galectin 3-binding protein levels.

One of ordinary skill in the art will appreciate that there are many techniques useful for measuring G3BP to practice the present invention. For example, G3BP levels can be measured using techniques such as flow cytometry and ELISA.

In one aspect, the present invention encompasses G3BP as a disease biomarker in general, with the specific disease best reflected by this marker to be determined. In another aspect, it can be used in diagnostic tests for cardiovascular disease, especially (vulnerable) plaque burden. In one aspect, G3BP is a useful marker for coronary artery disease. In another aspect, G3BP is useful as a surrogate end point in clinical studies. In yet another aspect, G3BP is useful as a biomarker of other disease and disorders, such as cancer, neurodegenerative diseases, renal diseases, liver disease, skin disease, and heart failure. In one aspect, the mRNA encoding G3BP is useful as a biomarker.

One of ordinary skill in the art will appreciate that multiple assays are available to detect and measure the biomarker proteins of the invention, as well as the mRNAs encoding the biomarker proteins.

In one embodiment, the present invention provides compositions and methods wherein determining the levels of G3BP is useful to identify subjects at risk, or at higher risk, for adverse events, as well as for establishing protocols and regimens for continued monitoring of the subject. For example, in one aspect, a subject with high levels of G3BP would need to be seen and monitored less frequently by a clinician than a subject found to have low levels of G3BP. That is, the frequency or intervals at which a subject is monitored would be based on the risk level of that subject for the adverse event, such as development or worsening of a cardiovascular associated disease or disorder. In one aspect, a low risk individual might be monitored once a year, while a subject at high risk might be monitored at least twice a year. One of ordinary skill in the art will appreciate that the schedule for such visits and monitoring may vary depending on parameters such as the age, sex, health and weight of the subject being monitored, as well as the particular level of G3BP in that subject. One of ordinary skill in the art will also understand that the levels of G3BP being used to recommend monitoring regimens do not necessarily need to be based on only high or low levels, but can be classified into additional levels as well. These levels can be determined using biological samples obtained from the subject using the assays described herein, or those known in the art, or new assays which are developed to measure G3BP levels.

In one embodiment, the biomarkers of the invention are useful for monitoring disease progression in cardiovascular associated diseases and disorders. In one aspect, the biomarker is G3BP.

In one embodiment, the biomarkers of the invention are useful for monitoring the effectiveness of treatment of diseases. In one aspect, the biomarkers are useful for monitoring therapeutic effects of treatment schemes or drugs, for example, statins, and other cholesterol-lowering drugs or anti-inflammatory schemes. In one aspect, the diseases and disorders being treated are cardiovascular associated diseases and disorders.

### BRIEF DESCRIPTION OF THE DRAWINGS

FIG. 1 graphically depicts the expression of mRNA encoding G3BP. PBMC: peripheral blood mononuclear cells; Monocytes: monocytes isolated as per methods, MCSF 6d, macrophages generated by incubation with M-CSF for 6 days (control for chemokines), MCP-1 and GRO-a are two pro-inflammatory chemokines and were added for the last 5 hours of the experiment; MCSF 8d: macrophages generated by incubation with M-CSF for 8 days (control for LDL conditions); MCSF oxLDL: macrophages grown in MCSF for 8 days, the last two days of which were supplemented with oxLDL; MCSF mmLDL and LDL are the corresponding conditions for mmLDL and native LDL; PF4: macrophages differentiated by incubation with PF4 as per methods, PF4+ oxLDL, oxLDL added for last 2 days (mean±SD). The ordinate represents expression level and the abscissa indicates the condition.

FIG. 2 graphically depicts Galectin-3-BP mRNA expression in macrophages generated with PF4 with and without oxLDL measured by gene chip (mean±SD). The ordinate represents expression and the ordinate represents the condition.

FIG. 3 graphically illustrates the effectiveness of G3BP as a biomarker. G3BP was found to detect CAD in asymptomatic (no angina) patients. G3BP levels were about 50% elevated (P<0.02). There was no significant difference in patients with angina (data not shown).

FIG. 4 graphically depicts the levels of G3BP in subjects receiving lipid lowering drugs with or without CAD. The ordinate represents G3BP levels in μg/ml. The abscissa indicates the class of subject. There was a trend for elevated G3BP in patients treated with lipid-lowering drugs (P<0.14).

FIG. 5 graphically demonstrates that G3BP is significantly higher in hypertensive patients (indicated as CAD) relative to those without hypertension (No CAD) (P<0.02). The ordinate represents G3BP levels in μg/ml. The abscissa indicates the class of subject.

FIG. 6 graphically suggests that there was a difference in G3BP levels in subjects with high (>150 mg/dl) versus low (<150 mg/dl) triglycerides (P=0.07). The ordinate represents G3BP levels in μg/ml. The abscissa indicates the class of subject.

FIG. 7 graphically suggests that G3BP levels are inversely related to total cholesterol levels (P<0.09). The ordinate represents G3BP levels in μg/ml. The abscissa indicates the class of subject (cholesterol<200 mg/dl or >200 mg/dl).

FIG. 8 graphically suggests that G3BP levels are inversely related to LDL cholesterol levels (P<0.02). The ordinate represents G3BP levels in μg/ml. The abscissa indicates the class of subject (LDL<120 mg/dl or >120 mg/dl).

FIG. 9 graphically depicts the serum levels of G3BP in coronary artery disease patients divided by endpoint. The combined endpoint was defined as the need for percutaneous or surgical revascularization and death up to 18 months after coronary angiography. Mann-Whitney non-parametric testing revealed a statistically significant difference (P<0.05).

FIG. 10 graphically depicts a receiver operator analysis of G3BP serum levels to predict combined outcome as defined in FIG. 9 in patients with coronary artery disease. The area under the curve is significant (P<0.05). The best cut-off in this population is 5.825 μg/ml with a likelihood ratio of 9.39.

FIG. 11 graphically depicts event-free survival in patients with coronary artery disease and G3BP serum levels below or above 5.825 μg/ml, which had been determined as cut-off by ROC analysis (FIG. 10). Events were defined as combined endpoint including the need for percutaneous or surgical revascularization and death up to 18 months after coronary angiography. Kaplan Meier survival analysis revealed a statistically significant difference between both patient groups (P<0.001).

### DETAILED DESCRIPTION OF THE INVENTION

### Abbreviations and Acronyms

AIM means Apoptosis Inhibitor in Macrophages
C4BP means Complement Component C4 Binding Protein
CAD means coronary artery disease
FCGBP means Fc fragment of IgG binding protein
FDR means false discovery rate
G3BP means Galectin 3-binding protein, also known as Mac2BP
HEM means heterogeneous error model
ICAT means Isotope-Coded Affinity Tag
LC/MS means liquid chromatography/mass spectrometry
LDL means low density lipoprotein
LPE means local pooled error
mmLDL means modified LDL
MPs means Microparticles
NE means not expressed
oxLDL means oxidized LDL
PAGE means Polyacrylamide gel electrophoresis
PBS means Phosphate buffered saline
PPP means Platelet-Poor Plasma
PRP means Platelet-Rich Plasma
SD means standard deviation
SDS means sodium dodecyl sulfate
SOM means self-organizing maps
vWF means von Willebrand Factor

### Definitions

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. For example, in one aspect, the term “about” is used herein to modify a numerical value above and below the stated value by a variance of 20%.

As used herein, the term “affected cell” refers to a cell of a subject afflicted with a disease or disorder, which affected cell has an altered phenotype relative to a subject not afflicted with a disease, condition, or disorder.

Cells or tissue are “affected” by a disease or disorder if the cells or tissue have an altered phenotype relative to the same cells or tissue in a subject not afflicted with a disease, condition, or disorder.

As used herein, “amino acids” are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

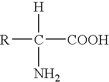

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group. The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “basic” or “positively charged” amino acid, as used herein, refers to amino acids in which the R groups have a net positive charge at pH 7.0, and include, but are not limited to, the standard amino acids lysine, arginine, and histidine.

As used herein, the term “biologically active fragments” or “bioactive fragment” of the polypeptides encompasses natural or synthetic portions of the full-length protein that are capable of specific binding to their natural ligand or of performing the function of the protein.

A “biomarker” is a specific biochemical in the body which has a particular molecular feature that makes it useful for measuring the progress of disease or the effects of treatment, or for measuring a process of interest.

A “compound,” as used herein, refers to a polypeptide, an isolated nucleic acid, or other agent used, identified, or isolated in the method of the invention.

As used herein, the term “conservative amino acid substitution” is defined herein as an amino acid exchange within one of the following five groups:

I. Small aliphatic, nonpolar, or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

The term “microparticle,” as used herein, refers to any protein containing particle less than 1 micron in diameter with a molecular weight of over 100,000 daltons. These include various lipoproteins and membrane vesicles released from cells.

As used herein, a “peptide” encompasses a sequence of 2 or more amino acid residues wherein the amino acids are naturally occurring or synthetic (non-naturally occurring) amino acids covalently linked by peptide bonds. No limitation is placed on the number of amino acid residues which can comprise a protein's or peptide's sequence. As used herein, the terms “peptide,” polypeptide,” and “protein” are used interchangeably. Peptide mimetics include peptides having one or more of the following modifications:

1. peptides wherein one or more of the peptidyl —C(O)NR— linkages (bonds) have been replaced by a non-peptidyl linkage such as a —CH₂₋carbamate linkage (—CH₂OC(O)NR—), a phosphonate linkage, a —CH₂₋sulfonamide (—CH₂—S(O)₂NR—) linkage, a urea (—NHC(O)NH—) linkage, a —CH₂-secondary amine linkage, or with an alkylated peptidyl linkage (—C(O)NR—) wherein R is C₁₋C₄ alkyl;

2. peptides wherein the N-terminus is derivatized to a —NRR₁ group, to a —NRC(O)R group, to a —NRC(O)OR group, to a —NRS(O)₂R group, to a —NHC(O)NHR group where R and R₁ are hydrogen or C₁₋C₄ alkyl with the proviso that R and R₁ are not both hydrogen;

3. peptides wherein the C terminus is derivatized to —C(O)R₂ where R₂ is selected from the group consisting of C₁₋C₄ alkoxy, and —NR₃R₄ where R₃ and R₄ are independently selected from the group consisting of hydrogen and C₁₋C₄ alkyl.

Synthetic or non-naturally occurring amino acids refer to amino acids which do not naturally occur in vivo but which, nevertheless, can be incorporated into the peptide structures described herein. The resulting “synthetic peptide” contains amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

“Plurality” means at least two.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups. As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “purified” relates to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

A “sample,” as used herein, refers preferably to a biological sample from a subject, including, but not limited to, normal tissue samples, diseased tissue samples, biopsies, blood, plasma, serum, saliva, feces, semen, tears, milk, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

The term “sign,” as used herein, refers to any abnormality indicative of a disease, disorder, or condition, discoverable on examination of the patient; an objective indication of disease, in contrast to a symptom, which is a subjective indication of disease.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

The term “standard,” as used herein, refers to something used for comparison. For example, a standard can be a known standard agent or compound which is administered or added to a control sample and used for comparing results when measuring said compound in a test sample. Standard can also refer to an “internal standard,” such as an agent or compound which is added at known amounts to a sample and is useful in determining such things as purification or recovery rates when a sample is processed or subjected to purification or extraction procedures before a marker of interest is measured.

A “subject” of analysis, diagnosis, or treatment is an animal. Such animals include mammals, preferably a human.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse and other observers.

### Embodiments of the Invention

In one embodiment, the present invention provides compositions and methods useful for diagnosing and monitoring the progression of cardiovascular associated diseases or disorders. In one aspect, the invention provides a biomarker, G3BP, for diagnosing and monitoring the progression of cardiovascular associated diseases or disorders. In one aspect, the present invention provides methods for isolating microparticles from plasma which has been depleted of platelets. In one embodiment, the present invention provides a method for identifying and analyzing microparticles isolated from plasma. In one aspect, the method provides for isolating microparticles from platelet-poor plasma. In one aspect, the method provides for identifying and analyzing biomarkers associated with microparticles.

In one aspect, the present invention provides the biomarker G3BP, or homologs or fragments thereof. In one aspect, the presence of a biomarker identified by the methods of the invention, or a difference in the level of the biomarker relative to a normal control level, is indicative of a disease, disorder, or condition. In one embodiment, the present invention provides diagnostic assays for diseases, disorders, and conditions using biomarkers identified by the methods of the invention.

The practice of the invention is not limited to only the techniques described herein for identifying or measuring G3BP levels. The present invention encompasses techniques for detecting and measuring G3BP based on using the whole sequence of G3BP and fragments of G3BP (such as SEQ ID NOs:1-13). These sequences are:

| | |
|---|---|
| SEQ ID NO: 1- | TIAYENK; |
| SEQ ID NO: 2- | YSSDYFQAPSDYR; |
| SEQ ID NO: 3- | ELSEALGQIFDAQR; |
| SEQ ID NO: 4- | SQLVYQSR; |
| SEQ ID NO: 5- | SDLAVPSELALLK; |
| SEQ ID NO: 6- | AVDTWSWGER; |
| SEQ ID NO: 7- | TLQALEFHTVPFQLLAR; |
| SEQ ID NO: 8- | LADGGATNQGR; |
| SEQ ID NO: 10- | GQWGTVCDNLWDLTDASVVCR; |
| SEQ ID NO: 11- | RIDITLSSVK; |
| SEQ ID NO: 12- | ASHEEVEGLVEK; |
| and | |
| SEQ ID NO: 13- | LASAYGAR. |

Various techniques available in the art of cellular and molecular biology and clinical diagnostics can be used to identify and measure G3BP. These include, but are not limited to, the use of antibodies directed against G3BP or specific fragments or regions of G3BP, various spectroscopy techniques, etc., as well as techniques to measure the levels of nucleic acids encoding G3BP, such as G3BP mRNA.

The invention is not limited to measuring G3BP levels in only the types of biological samples described herein.

It will be appreciated, of course, that the peptides may incorporate amino acid residues which are modified without affecting activity. For example, the termini may be derivatized to include blocking groups, i.e. chemical substituents suitable to protect and/or stabilize the N- and C-termini from “undesirable degradation,” a term meant to encompass any type of enzymatic, chemical or biochemical breakdown of the compound at its termini which is likely to affect the function of the compound, i.e. sequential degradation of the compound at a terminal end thereof.

The practice of the invention encompasses measuring changes in G3BP for any disease where a change in the level of G3BP is associated with the disease or disorder. In one aspect, the disease or disorder is a cardiovascular associated disease or disorder. In one aspect, the cardiovascular associated disease or disorder is coronary artery disease. In another aspect, the cardiovascular associated disease or disorder includes, but is not limited to, circulatory diseases induced or exasperated by an inflammatory response, such as ischemia, atherosclerosis, peripheral vascular disease, restenosis following angioplasty, inflammatory aortic aneurysm, vasculitis, stroke, spinal cord injury, congestive heart failure, hemorrhagic shock, ischemia/reperfusion injury, vasospasm following subarachnoid hemorrhage, vasospasm following cerebrovascular accident, pleuritis, pericarditis, and the cardiovascular complications of diabetes.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine arc also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein. The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art. Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide can be purified to remove contaminants. In this regard, it will be appreciated that the peptide will be purified to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-,C₈- or C₁₈— silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure peptide may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

The present invention is also directed to pharmaceutical compositions comprising the compounds of the present invention. More particularly, such compounds can be formulated as pharmaceutical compositions using standard pharmaceutically acceptable carriers, fillers, solublizing agents and stabilizers known to those skilled in the art.

### Examples

The invention is now described with reference to the following Examples and Embodiments. Without further description, it is believed that one of ordinary skill in the art can, using the preceding description and the following illustrative examples, make and utilize the present invention and practice the claimed methods. The following working examples therefore, are provided for the purpose of illustration only and specifically point out some embodiments of the present invention, and are not to be construed as limiting in any way the remainder of the disclosure. Therefore, the examples should be construed to encompass any and all variations which become evident as a result of the teaching provided herein.

### Example 1

Isolation of Platelets, Platelet-Derived Microparticles (MPs), and Plasma-Derived MPs.

Platelets and platelet-derived MPs were isolated as described. Briefly, human blood was collected by venipuncture into 1/10 volume of acid-citrate-dextrose (85 mM trisodium citrate, 83 mM dextrose, and 21 mM citric acid) solution. Platelet-rich plasma (PRP) was obtained by centrifugation at 110×g for 15 min. Platelets were pelleted by centrifugation at 710×g for 15 min. and the supernatant, platelet-poor plasma (PPP), was retained for isolation of plasma MPs (see below). The platelet pellet was washed three times, resuspended in 10 mL of Tyrode's buffer, and centrifuged one additional time at 110×g to remove remaining red blood cells or debris. To generate platelet-derived microparticles, ADP (10 μM final concentration) was added to the platelet suspension for 10 min. Platelets were removed by centrifugation (710×g for 15 min.) and platelet derived MPs were pelleted by centrifugation at 150,000×g for 90 min at 10° C.

Plasma-derived MPs were isolated by gel filtration chromatography followed by ultracentrifugation. Briefly, the platelet-poor plasma (PPP) generated above was centrifuged an additional two times to remove residual cells and cell debris at 710×g and 25° C. for 15 min. This plasma was then applied to a Sephacryl® S-500 HR (GE Healthcare, Piscataway, N.J.) gel filtration column and MP containing fractions were concentrated by ultracentrifugation at 150,000×g for 90 min. at 10° C.

Sample Preparation for Unlabeled Protein Analysis.

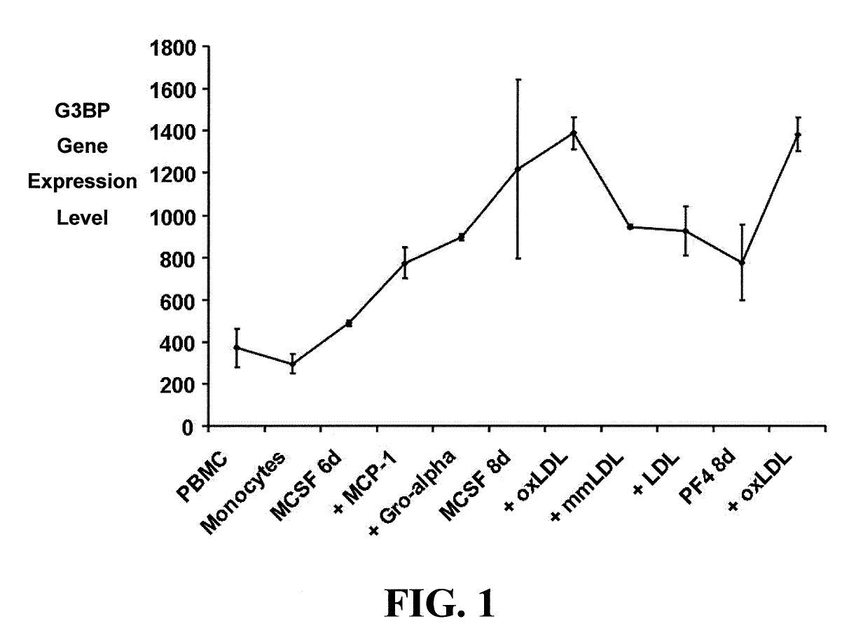

Platelet- and plasma-derived microparticle pellets were processed as described in Smalley et al., 2007, Thromb. Haemost., 97:67-80 (See FIG. 1A). MPs were resuspended in a minimal volume of PBS (phosphate buffered saline, pH 7.4) and a small aliquot was taken for protein analysis using the Micro BCA Protein Assay (Pierce Biotechnology, Inc., Rockford, Ill.). Forty microliters of plasma microparticles and equivalent amounts of protein from the platelet MPs, resuspended with PBS to 40 μL, were mixed with 10 μL of 5×SDS-PAGE loading buffer (0.5 M Tris, pH 6.8, 10% SDS, 38% glycerol, 0.1% bromophenol blue). The separate samples (50 μL each) were heated to 95° C. for 5 min., allowed to cool to room temperature, and centrifuged for 2 min. at 14,000 rpm prior to loading. Microparticle proteins were electrophoresed approximately 1 cm into a 7.5% acrylamide SDS-PAGE using a Mini-gel system (BioRad, Hercules, Calif.) at 150 V. The acrylamide gel section containing the proteins was cut out and placed in fixative (50% Methanol, 12% Acetic Acid, 0.05% formalin) for 2 hrs. The in-gel tryptic digestion of the lanes and the peptide extraction were performed as described. The extracted peptide solutions were lyophilized and reconstituted to 20 μL with 0.1% acetic acid for mass spectrometry analysis. A total of three sets of platelet- and plasma-derived MP peptides were generated and each of these samples was analyzed by LC/MS twice.

ICAT-Labeling, Electrophoresis, and Digestion, and Peptide Enrichment

Relative quantitation of proteins following ICAT labeling [3] was performed as described in Smalley et al., 2007, Thromb. Haemost., 97:67-80 (See FIG. 1B). Briefly, platelet- and plasma-derived microparticle pellets were resuspended in PBS (Phosphate buffered saline, pH 7.4) and protein concentration was determined. Solutions containing equivalent protein amounts of paired samples (plasma MP and platelet-derived MP) were lyophilized and resuspended in 1% SDS in denaturing buffer. Samples were labeled and processed using the ICAT labeling kit (Applied Biosystems, Foster City, Calif.) as instructed with the following modifications. The initial labeling reaction was done at ½ volume, protein amount, and ICAT reagent because of the low amount of protein obtained from each plasma MP preparation. The differentially-labeled proteins were mixed, applied to the gel, electrophoresed, and cut from the gel as described above except the loading buffer did not contain the reducing agent or SDS. The proteins were digested with trypsin, extracted from the gel, and processed through the avidin column as recommended by manufacturer. The samples were lyophilized and reconstituted to 20 μL with 0.1% acetic acid for mass spectrometry analysis. This procedure was repeated three times with plasma MPs labeled with the light ICAT reagent for two of these and labeled with the heavy ICAT reagent in the third.

Liquid Chromatography/Mass Spectrometry (LC/MS) and Protein Identification

Samples were loaded onto a 360 μm o.d.×75 μm i.d. microcapillary fused silica precolumn packed with irregular 5-20 μm C18 resin. After sample loading, the precolumn was washed with 0.1% acetic acid for 15 min. to remove any buffer salts or gel contaminants. The precolumn was then connected to a 360 μm o.d.×50 μm i.d. analytical column packed with regular 5 μm C18 resin constructed with an integrated electrospray emitter tip. Samples were gradient eluted with an 1100 series binary HPLC solvent delivery system (Agilent, Palo Alto, Calif.) directly into a Finnigan LTQ ion trap mass spectrometer (Thermo Electron Corp, San Jose, Calif.) at a flow rate of 60 nl/min. The HPLC stepwise gradient used was initially 100% A, 5% B at 5 min., 50% B at 220 min., 100% B at 240 min, and restored to 100% A at 280 min. (solvent A=0.1 M acetic acid, solvent B=70% acetonitrile in 0.1 M acetic acid). The LTQ mass spectrometer was operated in the data-dependent mode in which first an initial MS scan recorded the mass to charge (m/z) ratios of ions over the mass range 300-2000 Da, and then the 10 most abundant ions were automatically selected for subsequent collisionally-activated dissociation and an MS/MS spectrum recorded. All MS/MS data were searched against a human protein database downloaded from the National Library of Medicine NCBI website using the SEQUEST® program (Thermo Electron Corp.). For unlabeled peptides, a static modification of 57 Da for cysteine residues was employed in the search parameters. For ICAT-labeled peptides, a static modification of 227.127 was used for the light isotope label and an additional 9 Daltons for the heavy ICAT-labeled peptides. Peptide identifications were made using a first-pass filtering of standard criteria as previously described, including cross correlation values≧2.0 (+1 charge), 2.2 (+2 charge) and 3.5 (+3 charge) and all peptides must be fully tryptic. Protein assignments were only made if the protein had at least two or more MS/MS spectra passing the above criteria. Manual validation of at least one MS/MS spectrum per protein was performed for all proteins that were determined to be differentially expressed.

Comparative Analysis of Unlabeled Peptides Using Spectral Count.

For the unlabeled scheme (as in Smalley et al., 2007, Thromb. Haemost., 97:67-80, FIG. 1A), all MS/MS spectra not passing the first pass filter were eliminated. The number of spectra for each peptide was determined and the number of total proteins detected were calculated. If any protein had a spectral count of less than 2 for either the plasma MPs or the platelet MPs, it was eliminated from that group. Only proteins with an overall spectral count of 10 or greater were analyzed by this method. The ratio of spectra from the plasma MP versus the platelet MP was calculated, log 2 transformed, and then adjusted for an overall log score of 0.00 excluding vWF-containing peptides. The standard deviation (SD) of the log score was calculated and all proteins that were over 3 SD above (or below) the mean were considered to be enriched with “high confidence”. Proteins with log 2 scores between 2 and 3 SD away form the mean were possible candidates which should be examined further.

Comparative Analysis of ICAT-Labeled Peptides Using MSight®.

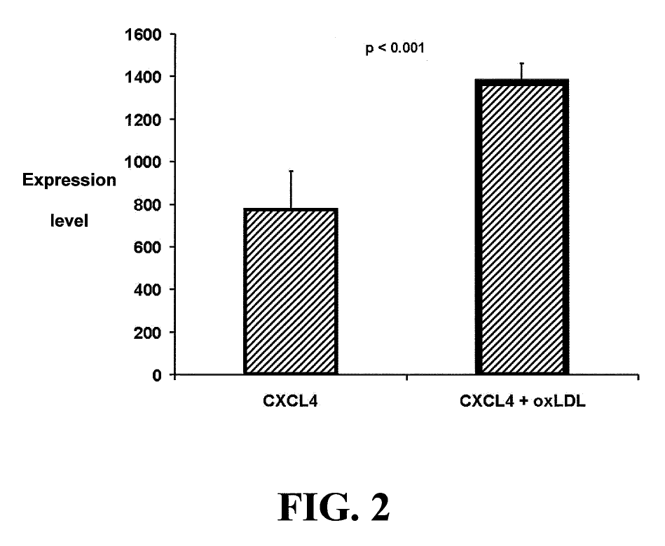

Comparative quantitation was performed using MSight®, freely available from the Swiss Institute of Bioinformatics website. Data files (.RAW) generated using XCalibur Software (Thermo Electron Corp.) were converted to mzXML files using ReaDW (Institute for Systems Biology, Seattle, Wash.). These files were imported into MSight®, and the peptides were manually quantified. The peptides were identified using SEQUEST® as described above. A sample representation of the MSight® display for a small portion of one of these runs is shown in FIG. 2 of Smalley et al. (2007, Thromb. Haemost., 97:67-80). Initially, peptides that were detected in all three ICAT analyses based on SEQUEST® results were quantified, if possible. Then, peptides with high ion intensities were quantified and linked backed to SEQUEST® results. Attempts to quantify peaks sometimes failed due to poor signal-to-noise ratios, overlapping peptides, and ambiguous identification of a given peak. Unless otherwise noted, ICAT quantitation results are only reported if good peak quantitation was possible in at least two of the three ICAT runs and labeled alternatively in these two runs. This led to the quantitation of 94 peptides. Utilizing the differential labeling, the ratios of the relative quantitation in plasma MPs versus platelet MPs were calculated, a log 2 transformation of these ratios was performed, and adjusted to generate an overall log 2 score of 0.00, excluding vWF. VWF was excluded because it was evident that there was a significant enrichment in the plasma MPs and due to the large number of peptides examined for this protein and the extent of enrichment, not omitting it would make it appear that most other proteins were enriched in the platelet MPs. To determine significance, the paired t-test was used to examine differences in peptide expression intensities between Plasma MPs and Platelet MPs. P<0.05 was used to identify differentially expressed peptides.

Affymetrix Gene Chip Analysis

Human blood was drawn from the antecubital veins of healthy blood donors and provided as buffy coats by the Virginia Blood Services (Richmond, Va.). The mononuclear fractions were pooled from four unidentified donors to decrease individual variations in monocytes. Mixed peripheral blood mononuclear cells (PBMCs) were isolated by Histopaque 1.077 (Sigma Diagnostics, Inc., St. Louis, Mo.). Following centrifugation, the mononuclear layer was removed and washed with PBS containing 0.02% ethylenediaminetetraacetate (EDTA). The pellet was resuspended in 1× H-lyse Buffer (R&D Systems Inc., Minneapolis, Minn.), and washed with wash buffer. PBMCs contain mainly monocytes and lymphocytes as well as platelets that tend to be associated with blood monocytes. From these PBMCs, monocytes were isolated using a negative selection monocyte isolation kit and LS columns (Miltenyi Biotec, Bergisch Gladbach, Germany). The purity of the isolated fraction was >97% as estimated by flow cytometry using anti-CD14.

Monocytes were cultured in Macrophage Serum-Free Medium (MSFM, Invitrogen, Carlsbad, Calif.) in the presence of 1% media supplement nutridoma-HU (Roche Molecular Biochemicals, Indianapolis, Ind.) and 100 nM M-CSF for 6 days, after which the cells showed the expected morphological signs of macrophage differentiation. These macrophages were incubated either with 100 nM MCP-1 (CCL2) or GROα (CXCL1) for 5 hours. Monocyte-derived macrophages (MDM) were also incubated with native LDL, oxidized LDL (oxLDL) or minimally modified LDL (mmLDL) (each at a concentration of 100 μg/ml) for 2 days to induce foam cell formation. Foam cell formation was verified by oil red O staining (FIG. 1) and by determining their cholesterol and cholesterol ester content. OxLDL and mmLDL were prepared from the same native LDL for each experiment as described. Control experiments were conducted on macrophages cultured in M-CSF without LDL for an additional 2 days. Two separate sets of monocytes were incubated with platelet factor-4 (CXCL4) (100 nM) for 6 days, another procedure known to induce macrophage differentiation, with and without oxLDL to induce foam cell formation. RNA was extracted from cells in all 11 conditions (table 1) and gene expression was measured in duplicates at the University of Virginia Gene Expression Core Facility using Affymetrix equipment.

Signal intensity values were obtained from the Affymetrix MicroArray Suite software (MAS 5.0). Of 22,283 probe sets on the HG-U133A chip, 78 internal control probes were removed and 22,215 probe sets representing 12,978 gene products were analyzed. Microarray gene expression intensities were normalized in order to ensure that all 22 array chips have the same inter-quartile ranges (IQR). In addition, they were log-transformed with base 2, which allows a natural interpretation as fold changes and transforms the right-skewed distribution closer to a normal distribution. While the log transformation enables a convenient interpretation of differential expression as fold changes, it is not a transformation that typically stabilizes variance. The variability of log-intensity measurements in oligonucleotide microarrays tends to decrease non-linearly with the increase in the mean expression intensity. This is in part due to common background noise at each spot of the microarray. At high intensity levels, this background noise is dominated by the expression intensity, while at low levels the background noise is a large component of the observed expression intensity.

The commonly used method of fold-change cutoff (for example, 2-fold) is not suitable for rigorous statistical analysis of gene expression, because at any given cutoff many genes with low levels of expression do not meet significance criteria, and other, highly expressed genes may miss the cutoff, although their change is really significant. For statistical analysis, an open source statistical software package R was used, which includes the local pooled error (LPE) test for differential expression discovery under two conditions, the heterogeneous error model (HEM) for differential expression discovery under multiple conditions, hierarchical clustering & heatmap analysis, and self-organizing maps (SOM), especially the last two widely used in microarray data analysis. The annotation information available from the Affymetrix website was used to identify the genes represented on the HG-U133A chip for the various classes of genes analyzed (see results). We eliminated non-expressed (within 2 SD from zero in all conditions) and housekeeping genes (not significantly regulated with false discovery rate FDR<0.05) as described below. We analyzed the regulated genes using LPE, HEM and heatmap analysis, and unbiased analysis of all regulated genes without prior knowledge such using hierarchical clustering analysis.

To identify genes (probe sets) that are not expressed in monocyte/macrophages and hence do not appear in any of the 11 conditions, we estimated baseline standard deviations (SD) by LPE as described below. Probe sets with expression values within 2SD from zero in all 11 conditions were considered not expressed (NE); hence, they were eliminated from further analysis (see results). HK genes are defined as genes that do not change their expression when cells undergo phenotypic changes. Of the 11 conditions, the first two conditions (PBMC and monocytes) were not used to find HK genes because the gene expression changes between monocytes and macrophages dominated other changes of many genes. We broadly defined housekeeping genes for monocyte/macrophages as genes that showed similar expression in all conditions based on HEM scores. All genes with a false discovery rate of greater than 0.05 by HEM were considered unchanged over the 9 conditions studied. This eliminated 16,783 genes from further analysis.

The LPE test was used to investigate differentially expression under two conditions because it is statistically powerful in identifying differentially expressed genes with low-replicated microarray data, e.g., duplicate or triplicate. LPE pools probe sets with similar expression levels were used to estimate baseline variances, which improves individual gene variance estimation with low replication. The LPE test provides a statistic for each probe set and the absolute value of the LPE-statistic is larger for more significantly differentially expressed probe set. HEM is designed to investigate differential expression in microarray experiments comparing multiple conditions, taking advantages of the error pooling power of LPE as its prior specifications. HEM captures heterogeneous error variability of microarray data, so that it enabled us to reliably identify differentially expressed genes with a significantly higher statistical power from the macrophage microarray data with limited replication (duplicates). In addition, a false discovery rate (FDR) was calculated to discover probe sets differentially expressed with FDR<0.05.

Here, we identify G3BP as a macrophage-derived protein. Beyond the biomarkers identified so far, we assert that this technique can be used to identify additional biomarkers from human blood. We further assert that some of these biomarkers are useful for identifying disease states in patients, monitor progression of disease and response to treatment.

Table 1 shows expression of G3BP in plasma-derived but not platelet-derived microparticles.

TABLE 1

| | | Plasma | | Platelet | | |
|---|---|---|---|---|---|---|
| | | MP | Plasma | MP | Platelet | |
| Protein | Accession | total | MP | total | MP | Spectral |
| Name | No. | scans | Peptides | scans | Peptides | Count |
| galectin 3 | 5031863 | 68 | 18 | 0 | 0 | 68 |
| binding | | | | | | |
| protein | | | | | | |

FIG. 1 depicts the expression of mRNA encoding G3BP. PBMC, peripheral blood mononuclear cells; Monos, monocytes isolated as per methods, MCSF 6d, macrophages generated by incubation with M-CSF for 6 days (control for chemokines), MCP-1 and GRO-a are two pro-inflammatory chemokines and were added for the last 5 hours of the experiment; MCSF 8d, macrophages generated by incubation with M-CSF for 8 days (control for LDL conditions); MCSF oxLDL: macrophages grown in MCSF for 8 days, the last two days of which were supplemented with oxLDL; MCSF mmLDL and LDL are the corresponding conditions for mmLDL and native LDL; PF4 mono: macrophages differentiated by incubation with PF4 as per methods, PF4 oxLDL, oxLDL added for last 2 days (mean±SD).

Table 2 shows the significance levels (by LPE as per methods) and the false discovery rates (by LPE as per methods) for altered G3BP in each of the conditions. Statistically significant differences highlighted in yellow.

TABLE 2

| | | Monos | | | | | | MCSF | | MCSF | | MCSF | | PF4 | |
|---|---|---|---|---|---|---|---|---|---|---|---|---|---|---|---|
| PBMC | | vs. | | MCSF | | MCSF | | 8 d vs. | | 8 d vs. | | 8 d vs. | | mono | |
| vs. | | MCSF | | 6 d vs. | | 6 d vs. | | MCSF | | MCSFS | | MCSF | | vs. PF4 | |
| Monos | | 6 d | | MCP-1 | | Gro-a | | oxLDL | | mmLDL | | LDL | | oxLDL | |
| LPE | FDR | LPE | FDR | LPE | FDR | LPE | FDR | LPE | FDR | LPE | FDR | LPE | FDR | LPE | FDR |
| 4.42 | 0.00 | 3.85 | 0.00 | −1.35 | 0.54 | 0.27 | 0.76 | −2.03 | 0.45 | 0.80 | 0.84 | −2.17 | 0.23 | −3.30 | 0.01 |

FIG. 2 depicts G3BP mRNA expression with and without oxLDL measured by gene chip (mean±SD).

Other methods useful in the practice of the invention can be found in PCT application number 11935048 “Methods for identifying and analyzing biomarkers from plasma-derived microparticles,” filed Nov. 5, 2007.

### Bibliography for Example 1

Garcia B A, Smalley D M, Cho H, Shabanowitz J, Ley K, Hunt D F. The platelet microparticle proteome. J Proteome Res. 2005; 4:1516-21.

Shevchenko A, Wilm M, Vorm O, Mann M. Mass spectrometric sequencing of proteins silver-stained polyacrylamide gels. Anal Chem. 1996; 68: 850-858.

U.S. Pat. No. 6,670,194, Aebersold , et al. Dec. 30, 2003: Rapid quantitative analysis of proteins or protein function in complex mixtures

Washburn M P, Wolters D, Yates J R, 3rd. Large-scale analysis of the yeast proteome by multidimensional protein identification technology. Nat Biotechnol. 2001; 19: 242-247.

Palagi P M, Walther D, Quadroni M, Catherinet S, Burgess J, Zimmermann-Ivol C G, Sanchez J C, Binz P A, Hochstrasser D F, Appel R D. MSight: an image analysis software for liquid chromatography-mass spectrometry. Proteomics. 2005; 5: 2381-2384.

Smalley D F, Root, K E, Cho, H, Ross M, Ley K (2007). Proteomic Discovery of 21 Proteins Expressed in Human Plasma-derived but not Platelet-derived Microparticles. Thrombosis and Hemostasis 97: 67-80.

Cho, H., Shashkin, P., Gleissner, C., Dunson, D., Jain, N., Lee, J., Miller, Y., Ley, K. (2007). Induction of dendritic cell phenotype during foam cell formation. Physiol. Genomics, 29:149-160.

### Example 2

Methods

Patients underwent coronary catheterization and angiography to determine coronary artery disease (CAD) by the accepted gold standard method. G3BP was analyzed by ELISA in serum from 65 individuals (17 no CAD, 48 CAD). More recent studies have included additional patients.

Results

a) G3BP as a Diagnostic Marker

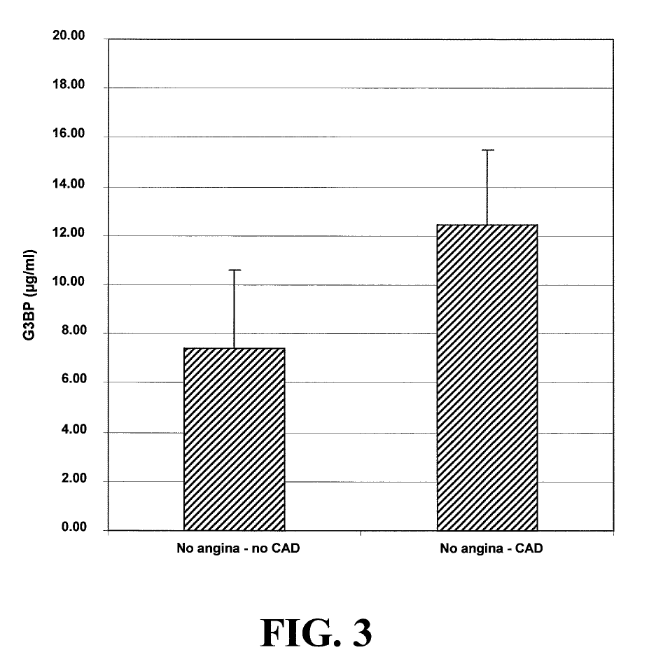

G3BP was found to detect CAD in asymptomatic (no angina) patients. G3BP levels were about 50% elevated (P<0.02) (see FIG. 3). There was no significant difference in patients with angina (data not shown).

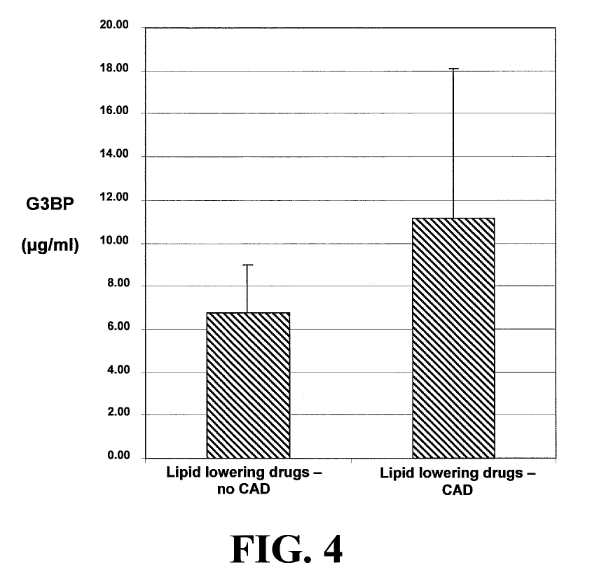

There was a trend for elevated G3BP in patients treated with lipid-lowering drugs (P<0. 14) (see FIG. 4). Larger patient numbers may make this difference significant. There was no difference in patients not on lipid-lowering drugs (data not shown).

b) Correlation with Demographic and Clinical Parameters

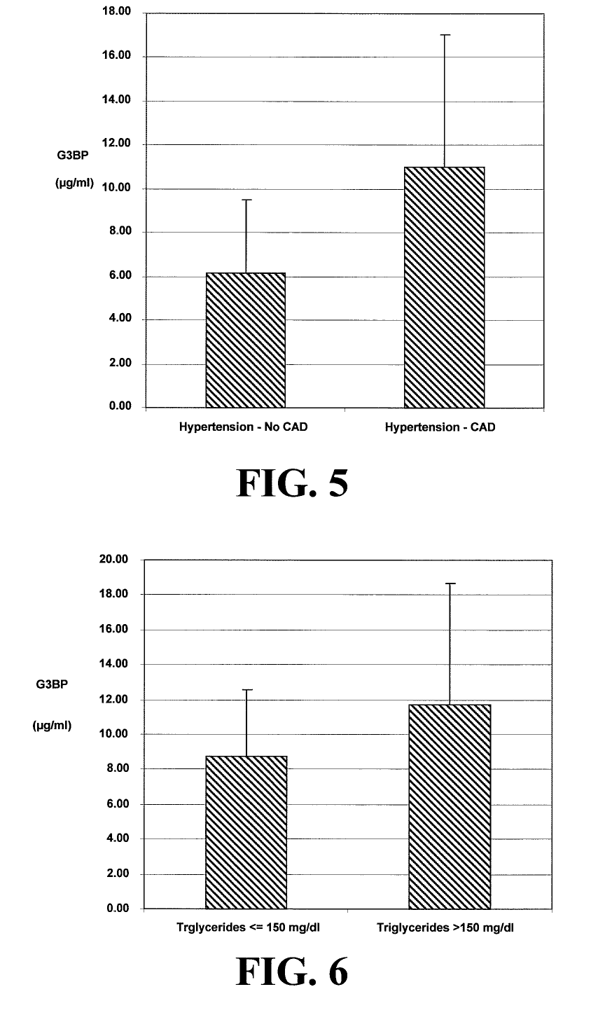

G3BP levels were not significantly related to: race, gender, heart rate, diabetes, family history, previous myocardial infarction, previous CABG, chronic heart failure, PTCA, future CABG, angina, but G3BP is significantly higher in hypertensive patients (P<0.02) (see FIG. 5). There was also a notable difference in triglycerides in early preliminary results (see FIG. 6; P=0.07). More recent results with a larger cohort suggest that this difference is due to one outlier and completely disappeared when this value was excluded.

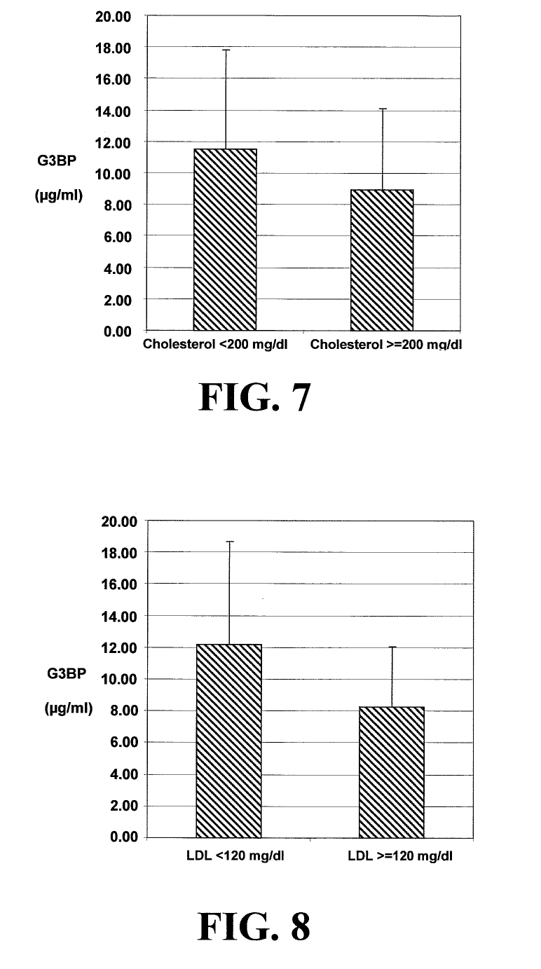

Preliminary results suggested that G3BP may be inversely related to total cholesterol levels (P<0.09) (see FIG. 7). Other preliminary results also suggested that G3BP may be inversely related to LDL cholesterol levels (P<0.02) (see FIG. 8). The preliminary results suggested that it could be a valuable additional biomarker in that it does not measure the risk measured by LDL-cholesterol. However, these results were not confirmed in the larger cohort.

Some specific peptides that are found in the amino acid sequence of G3BP were identified by mass spectrometry repeatedly with a high degree of confidence (see Table 3).

TABLE 3

| | |
|---|---|
| SEQ ID NO: 1- | TIAYENK |
| SEQ ID NO: 2- | YSSDYFQAPSDYR |
| SEQ ID NO: 3- | ELSEALGQIFDAQR |
| SEQ ID NO: 4- | SQLVYQSR |
| SEQ ID NO: 5- | SDLAVPSELALLK |
| SEQ ID NO: 6- | AVDTWSWGER |
| SEQ ID NO: 7- | TLQALEFHTVPFQLLAR |
| SEQ ID NO: 8- | LADGGATNQGR |
| SEQ ID NO: 9- | STHTLDLSR |
| SEQ ID NO: 10- | GQWGTVCDNLWDLTDASVVCR |
| SEQ ID NO: 11- | RIDITLSSVK |
| SEQ ID NO: 12- | ASHEEVEGLVEK |
| SEQ ID NO: 13- | LASAYGAR |

Without wishing to be bound by any particular theory, it is hypothesized herein that any of these peptides or any combination of these peptides may be of diagnostic value for specific cardiovascular or other diseases as listed in the disclosure. It is possible that G3BP may be fragmented by proteolytic cleavage or other processes that may result in a higher representation of these peptides. Specific ELISAs or other diagnostic assays may be developed that detect one or more of these peptides, or protein fragments containing these peptides. This is in addition to other assays that detect the intact G3BP or messenger RNA for G3BP.

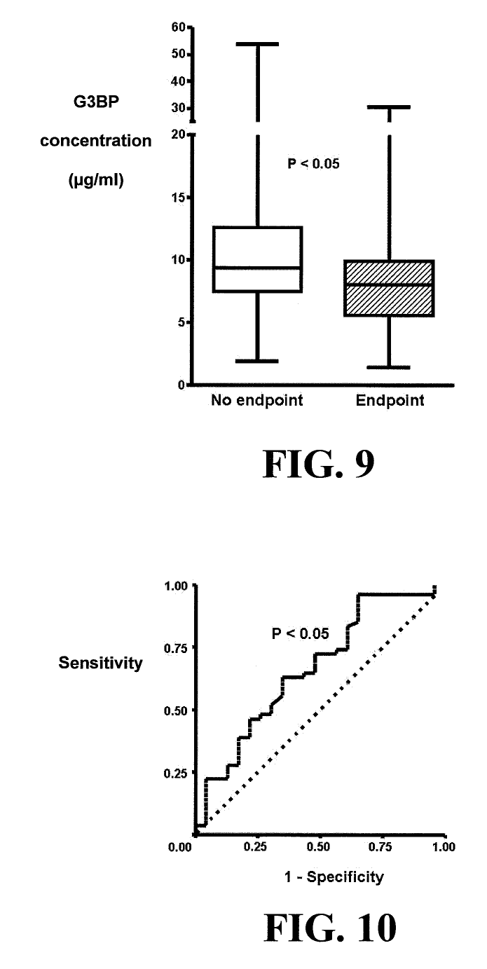

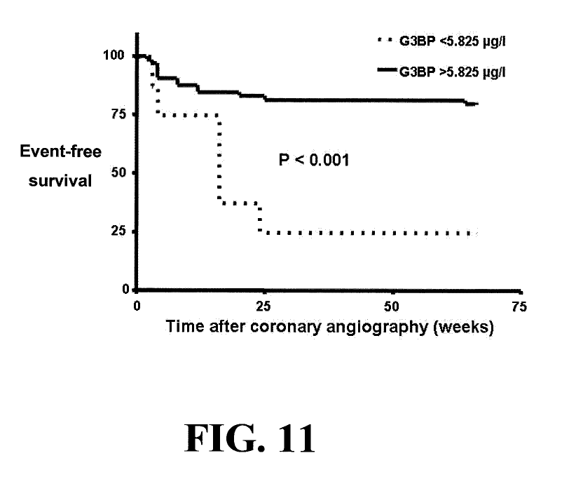

See also FIGS. 1-11.

### Bibliography for Example 2

Koths K, Taylor E, Halenbeck R, Casipit C, Wang A. Cloning and characterization of a human Mac-2-binding protein, a new member of the superfamily defined by the macrophage scavenger receptor cysteine-rich domain. J. Biol. Chem. Jul. 5, 1993; 268(19):14245-14249.

Inohara H, Akahani S, Koths K, Raz A. Interactions between galectin-3 and Mac-2-binding protein mediate cell-cell adhesion. Cancer Res. Oct. 1, 1996; 56(19):4530-4534.

Muller S A, Sasaki T, Bork P, Wolpensinger B, Schulthess T, Timpl R, Engel A, Engel J. Domain organization of Mac-2 binding protein and its oligomerization to linear and ring-like structures. J Mol Biol. Aug. 27, 1999; 291(4):801-813.

Grassadonia A, Tinari N, Iurisci I, Piccolo E, Cumashi A, Innominato P, D'Egidio M, Natoli C, Piantelli M, Iacobelli S. 90K (Mac-2 BP) and galectins in tumor progression and metastasis. Glycoconj J. 2004; 19(7-9):551-556.

Fornarini B, D'Ambrosio C, Natoli C, Tinari N, Silingardi V, Iacobelli S. Adhesion to 90K (Mac-2 BP) as a mechanism for lymphoma drug resistance in vivo. Blood. Nov. 1, 2000; 96(9):3282-3285.

Fusco O, Querzoli P, Nenci I, Natoli C, Brakebush C, Ullrich A, Iacobelli S. 90K (MAC-2 BP) gene expression in breast cancer and evidence for the production of 90K by peripheral-blood mononuclear cells. Int J Cancer. Feb. 20, 1998; 79(l):23-26.

Kalayci O, Birben E, Tinari N, Oguma T, Iacobelli S, Lilly C M. Role of 90K protein in asthma and TH2-type cytokine expression. Ann Allergy Asthma Immunol. November 2004; 93(5):485-492.

Smalley D F, Root, K E, Cho, H, Ross M, Ley K (2007). Proteomic Discovery of 21 Proteins Expressed in Human Plasma-derived but not Platelet-derived Microparticles. Thrombosis and Hemostasis 97: 67-80 (galectin-3-binding protein mentioned as one of 26 proteins)

Other methods which were used but not described herein are well known and within the competence of one of ordinary skill in the art of cell biology, molecular biology, and clinical medicine. The invention should not be construed to be limited solely to the assays and methods described herein, but should be construed to include other methods and assays as well. One of skill in the art will know that other assays and methods are available to perform the procedures described herein.

Headings are included herein for reference and to aid in locating certain sections. These headings are not intended to limit the scope of the concepts described therein under, and these concepts may have applicability in other sections throughout the entire specification. The disclosures of each and every patent, patent application, and publication cited herein are hereby incorporated herein by reference in their entirety. While this invention has been disclosed with reference to specific embodiments, it is apparent that other embodiments and variations of this invention may be devised by the previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Accordingly, the present invention is not intended to be limited to the embodiments shown herein but is to be accorded the widest scope consistent with the principles and novel features disclosed herein.

What is claimed is:

1. A method of diagnosing a cardiovascular associated disease or disorder in a test subject, said method comprising obtaining a biological sample from said test subject, and comparing the level of galectin 3-binding protein in said biological sample with the level of galectin 3-binding protein in an otherwise identical biological sample from a control subject without said cardiovascular associated disease or disorder, wherein a different level of galectin 3-binding protein in said test subject, compared with the level of galectin 3-binding protein in said biological sample from said control subject, is an indication that said test subject has a cardiovascular associated disease or disorder, thereby diagnosing a cardiovascular associated disease or disorder in a test subject.

2. The method of claim 1, wherein said cardiovascular associated disease or disorder is selected from the group consisting of coronary artery disease, circulatory disease exacerbated by ischemia, atherosclerosis, peripheral vascular disease, restenosis following angioplasty, surgical revascularization, inflammatory aortic aneurysm, vasculitis, stroke, spinal cord injury, congestive heart failure, cardiomyopathy, hemorrhagic shock, ischemia/reperfusion injury, vasospasm following subarachnoid hemorrhage, vasospasm following cerebrovascular accident, pleuritis, pericarditis, and the cardiovascular complications of diabetes.

3. The method of claim 2, wherein said cardiovascular associated disease or disorder is coronary artery disease.

4. The method of claim 2, wherein said test subject is a human.

5. The method of claim 2, wherein said test subject is at risk for said cardiovascular associated disease or disorder.

6. The method of claim 2, wherein said test subject is asymptomatic for said cardiovascular associated disease or disorder.

7. The method of claim 2, wherein said sample is selected from the group consisting of tissue, cells, blood, plasma, serum, tears, saliva, feces, semen, milk, sweat, and urine.

8. The method of claim 7, wherein said sample is plasma.

9. The method of claim 8, wherein said plasma is processed to obtain plasma-derived microparticles.

10. The method of claim 1, further wherein at least one other biomarker associated with a cardiovascular associated disease or disorder is measured.

11. The method of claim 1, wherein galectin 3-binding protein levels are measured using a technique selected from the group consisting of flow cytometry and ELISA.

12. The method of claim 1, wherein said change in galectin 3-binding protein levels is an increase in galectin 3-binding protein levels.

13. The method of claim 1, wherein said change in galectin 3-binding protein levels is a decrease in galectin 3-binding protein levels.

14. A method of monitoring a subject at risk for development of or worsening of a cardiovascular associated disease or disorder, said method comprising:
a) measuring the level of galectin 3-binding protein in a first biological sample obtained from said subject to determine an initial level of galectin 3-binding protein;
b) assessing the risk level of said subject for development of or worsening of a cardiovascular associated disease or disorder based on the level of galectin 3-binding protein; and
c) monitoring the subject at intervals based on the risk level assessed for said subject by measuring the levels of galectin 3-binding protein at said intervals,
thereby monitoring a subject at risk for development of or worsening of a cardiovascular associated disease or disorder.

15. A method of monitoring the treatment of a subject previously diagnosed with a cardiovascular associated disease or disorder, said method comprising:
a) measuring the level of galectin 3-binding protein in a first biological sample obtained from said subject to determine an initial level of galectin 3-binding protein;
b) treating said cardiovascular associated disease or disorder;
c) measuring the level of galectin 3-binding protein in a second otherwise identical biological sample obtained from said subject during or after said treatment;
d) comparing the level of galectin 3-binding protein in said first biological sample with the level of galectin 3-binding protein in said second otherwise identical biological sample obtained from said subject during or after said treatment; and
e) correlating any change in the level of galectin 3-binding protein in said second otherwise identical biological sample with the effectiveness of said treatment,
thereby monitoring the treatment of said subject previously diagnosed with said cardiovascular associated disease or disorder.

16. The method of claim 15, wherein said cardiovascular associated disease or disorder is selected from the group consisting of coronary artery disease, circulatory disease exacerbated by ischemia, atherosclerosis, peripheral vascular disease, restenosis following angioplasty, surgical revascularization, inflammatory aortic aneurysm, vasculitis, stroke, spinal cord injury, congestive heart failure, cardiomyopathy, hemorrhagic shock, ischemia/reperfusion injury, vasospasm following subarachnoid hemorrhage, vasospasm following cerebrovascular accident, pleuritis, pericarditis, and the cardiovascular complications of diabetes.

17. The method of claim 16, wherein said cardiovascular associated disease or disorder is coronary artery disease.

18. The method of claim 16, wherein said galectin 3-binding protein levels are measured more than once following the measurement of the initial levels of galectin 3-binding protein.

19. The method of claim 16, wherein said treatment is with a cholesterol lowering drug.

20. A method of monitoring the progression of a cardiovascular associated disorder in a subject previously diagnosed with a cardiovascular associated disease or disorder, said method comprising:
a) measuring the level of galectin 3-binding protein in a first biological sample obtained from said subject to determine an initial level of galectin 3-binding protein;
b) measuring the level of galectin 3-binding protein in a second otherwise identical biological sample obtained from said subject at a later point in time,
d) comparing the level of galectin 3-binding protein in said first biological sample with the level of galectin 3-binding protein in said second otherwise identical biological sample obtained from said subject; and
e) correlating any change in the level of galectin 3-binding protein in said second otherwise identical biological sample with the progression of said disease or disorder,
thereby monitoring the progression of said cardiovascular associated disease or disorder in said subject.

21. The method of claim 20, wherein said galectin 3-binding protein n levels are measured more than once following the measurement of the initial levels of galectin 3-binding protein.


SEQUENCE LISTING (13 sequences)

SEQ ID NO: 1 - protein, 7 aa, homo sapiens
TIAYENK

SEQ ID NO: 2 - protein, 13 aa, homo sapiens
YSSDYFQAPSDYR

SEQ ID NO: 3 - protein, 14 aa, homo sapiens
ELSEALGQIFDAQR

SEQ ID NO: 4 - protein, 8 aa, homo sapiens
SQLVYQSR

SEQ ID NO: 5 - protein, 13 aa, homo sapiens
SDLAVPSELALLK

SEQ ID NO: 6 - protein, 10 aa, Homo sapiens
AVDTWSWGER

SEQ ID NO: 7 - protein, 17 aa, homo sapiens
TLQALEFHTVPFQLLAR

SEQ ID NO: 8 - protein, 11 aa, homo sapiens
LADGGATNQGR

SEQ ID NO: 9 - protein, 9 aa, homo sapiens
STHTLDLSR

SEQ ID NO: 10 - protein, 21 aa, homo sapiens
GQWGTVCDNLWDLTDASVVCR

SEQ ID NO: 11 - protein, 10 aa, homo sapiens
RIDITLSSVK

SEQ ID NO: 12 - protein, 12 aa, homo sapiens
ASHEEVEGLVEK

SEQ ID NO: 13 - protein, 8 aa, homo sapiens
LASAYGAR